(19) United States
(12) Patent Application Publication
(10) Pub. No.: US 2015/0327972 A1
(43) Pub. Date: Nov. 19, 2015

(54) SOFT TISSUE AUGMENTATION THREADS AND METHODS OF USE THEREOF

(71) Applicant: Allergan Industrie SAS, Pringy (FR)

(72) Inventors: Kenneth N. Horne, San Francisco, CA (US); Jayakumar Rajadas, Cupertino, CA (US); Geoffrey C. Gurtner, Stanford, CA (US); Naveen Jayakumar, Cupertino, CA (US)

(21) Appl. No.: 14/791,147

(22) Filed: Jul. 2, 2015

Related U.S. Application Data
(63) Continuation of application No. 13/880,502, filed on Jul. 3, 2013, now abandoned.
(60) Provisional application No. 61/405,175, filed on Oct. 20, 2010.

(51) Int. Cl. A61F 2/00; A61L 17/04

(52) U.S. Cl. CPC A61F 2/0059; A61L 17/04

(57) ABSTRACT

This disclosure relates generally to soft tissue augmentation threads, methods of making such threads and uses thereof, for example, in aesthetic applications (e.g., facial contouring, soft tissue augmentation products), surgery (e.g., sutures), drug delivery, negative pressure wound therapy, moist wound dressing, and the like.

### CROSS-REFERENCE TO RELATED APPLICATIONS

This is a continuation of U.S. patent application Ser. No. 13/880,502, filed on Jul. 3, 2013 which is a national stage application of PCT/US11/56182 filed Oct. 13, 2011 which claims the benefit under 35 U.S.C. §119(e) of U.S. Provisional Application 61/405,175, filed on Oct. 20, 2010, each of which is hereby incorporated by reference in its entirety.

### FIELD

This disclosure relates generally to soft tissue augmentation threads, methods of making such threads and uses thereof, for example, in aesthetic applications (e.g., facial contouring, soft tissue augmentation products), surgery (e.g., sutures), drug delivery, negative pressure wound therapy, moist wound dressing, and the like.

### BACKGROUND

Many common soft tissue augmentation products which are injected into the treatment site as a liquid or a gel, such as Restylane® (hyaluronic acid), Juvaderm® (hyaluronic acid) Radiesse® (calcium hydroxyl apatite), Sculptra® (poly-L-lactic acid) and Perlane® (hyaluronic acid), are capable of ingression and/or causing unsightly “lumps” which are painful to treat. Further, these gels will occupy the space of least resistance which makes its use in many applications (e.g., treatment of fine wrinkles) problematic as the gel will often ingress into unintended spatial areas rendering the cosmetic procedure difficult and possibly even dangerous, as these soft tissue augmentation products are not recommended for use around the eyes as mobility from the injection site can cause blindness, tissue necrosis, and in rare cases even stroke. Clinicians also find performing lip augmentations using these fillers time consuming, and patients find treatments in this area so painful that nerve blocks are routinely performed.

Accordingly, there is a need for new compositions and physical forms of soft tissue augmentation products which can be dispensed uniformly to specific locations regardless of tissue resistance, and without the risk of migration. Such soft tissue augmentation products should be biocompatible and, preferably, biodegradable. Such new forms will have particular uses, for example, in aesthetic and surgical applications, drug delivery, wound therapy and wound dressing.

### SUMMARY

Disclosed herein are soft tissue augmentation threads and methods for making the same. The threads are comprised of one or more biocompatible polymers, wherein at least a portion of which is non-peptidic, self-swellable or self-expandable, and carbohydrate based.

The exact nature of the soft tissue augmentation thread is not critical. Rather, the criticality of the soft tissue augmentation thread is manifested in one or more of the following: improved tensile strength, reduced biodegradation, improved ability to assist in regeneration and the like. An improved ability to promote regeneration and/or tissue repair in vivo is contemplated by forming a scaffold-like structure in the body for collagen deposition. This tissue repair could prolong the “filler” effects of the thread when used to treat or fill a wrinkle or provide facial contouring in vivo far beyond the half-life of the soft tissue augmentation thread.

In certain embodiments, the present disclosure is directed to a soft tissue augmentation product thread comprised of one or more biocompatible polymers, wherein at least a portion of which is non-peptidic, self-swellable or self-expandable, and carbohydrate based, and further wherein at least a portion of the biocompatible polymer is cross-linked. In one embodiment, the thread is non-compressible also.

In certain aspects, the thread is substantially cylindrical, substantially D-shaped, or substantially ribbon shaped.

The biocompatible polymers to be used in the present disclosure form a gel under aqueous conditions. This gel form can then be converted by the methods described herein to provide the novel threads described herein. In one process embodiment, an aqueous gel composition comprising one or more biocompatible polymers is dried under non-denaturing conditions, preferably ambient conditions, to provide a dry thread. In some embodiments, it is contemplated that other forms of drying, such as submersing in solvents, freezing, lyophilization, and heating, can be used to provide the threads of the invention. In some embodiments, it is desirable to cross-link the biocompatible polymer. Accordingly, in one process embodiment, an aqueous gel composition comprising one or more biocompatible polymers and a cross-linking agent is dried under denaturing conditions, preferably ambient conditions, to provide a dry thread.

In one of its method embodiments, there is provided a method of treating a wrinkle in a subject in need thereof. In such an aspect, the thread is inserted into the skin of a patient adjacent to or under the wrinkle. The thread is then applied under the wrinkle thereby treating the wrinkle. In one embodiment, upon exposure to body fluids or by manually hydrating, the thread will expand upon hydration and such expansion is typically sufficient to fill-in the wrinkle. It is advantageous to have a thread expand upon hydration because the invasiveness of the insertion profile is minimized, however, threads designed to not expand can also be used to treat the wrinkle.

In another embodiment, the disclosure is directed to providing facial contouring in a subject in need thereof. In this embodiment, the thread is inserted into the skin at or adjacent to the desired treatment location, e.g., the lips, the nasolabial fold, the tear trough, etc. The thread is then applied thereby providing facial contouring. In one embodiment, a thread is applied to various planes of the dermal tissue. In one embodiment, several threads can be placed generally parallel to each other and additional threads places in a generally perpendicular direction with respect to the first set of parallel threads thereby forming a mesh structure whose aggregate effect is to contour a larger defect or a more widespread defect, such as the tear trough or the infraorbital region of the eye.

Also encompassed by this disclosure is a kit of parts comprising the thread. In some embodiments, the kit further comprises a means for delivering the thread. The means for delivery can either be a syringe or a needle.

In still other aspects, the threads described herein can be used as adhesion barriers, wound dressings including negative pressure wound dressings, sutures, and the like. Further provided are methods of using the threads described herein for example, in surgery, ophthalmology, wound closure, drug delivery, and the like.

### BRIEF DESCRIPTION OF THE DRAWINGS

The disclosure is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 illustrates a thread attached to the proximal end of a needle, in its entirety (N=needle; T=thread).

FIGS. 2A-2B show a needle attached to the thread (N=needle; T=thread). FIG. 2A illustrates a close-up view of a thread inserted into the inner-diameter of a needle; and FIG. 2B illustrates a close-up view of the proximal end of a solid needle with the thread overlapping the needle.

FIGS. 3A-3F show treatment of a wrinkle FIG. 3A illustrates a fine, facial wrinkle in the peri-orbital region of a human; FIG. 3B illustrates a needle and thread being inserted into the skin of the wrinkle at the medial margin; FIG. 3C illustrates the needle being adjusted to traverse beneath the wrinkle; FIG. 3D illustrates the needle exiting at the lateral margin of the wrinkle; FIG. 3E illustrates the needle having pulled the thread into the location it previously occupied beneath the wrinkle; and FIG. 3F illustrates the thread implanted beneath the wrinkle, with excess thread having been cut off.

FIGS. 4A-4F show treatment of baldness. FIG. 4A illustrates a top-down view of a male with typical male-pattern baldness; FIG. 4B illustrates where hair re-growth is desired, taking hairlines into consideration; FIG. 4C illustrates a curved needle with attached thread being inserted into one imaginary line where hair re-growth is desired; FIG. 4D illustrates the needle traversing the imaginary line, and exiting the skin; FIG. 4E illustrates the needle pulled through distally, pulling along the thread into the desired location; and FIG. 4F illustrates scissors being used to cut excess thread.

FIGS. 5A-5C show treatment of a wrinkle FIG. 5A illustrates a cross-sectional view of a fold or a wrinkle; FIG. 5B illustrates a thread implanted beneath a wrinkle that is not yet hydrated; and FIG. 5C illustrates a thread implanted beneath a wrinkle that is fully hydrated and has flattened the surface appearance of the wrinkle.

FIGS. 6A-6D show treatment of a tumor. FIG. 6A illustrates a human pancreas with a tumor; FIG. 6B illustrates a curved needle with a thread attached thereto; FIG. 6C illustrates a curved needle traversing the tumor within the pancreas; and FIG. 6D illustrates the end-result of repeated implantations of thread.

FIGS. 7A-7C show a nipple reconstruction. FIG. 7A illustrates multiple layers of concentric coils of thread, shaped to represent a human nipple; FIG. 7B illustrates the implant of FIG. 7A in cross-section; and FIG. 7C illustrates how an implant of coiled thread would be used for nipple reconstruction.

FIG. 8 illustrates how a needle and thread could be used to place a thread in a specific, linear location to promote nerve or vessel regrowth in a specific line.

FIGS. 9A-9B show placement of threads in a relatively parallel orientation for facial contouring in the tear trough (Thread 1, 2, 3, 4, 5, and 6). This figure also shows placement of the thread for facial contouring of the nasolabial fold (Thread 7 and 8). FIG. 9B shows an alternative placement of the threads for facial contouring in the tear trough (Thread 1, 2, 3, 4, 5, 6, 7, and 8). This figure also shows the potential placement of the threads in the nasolabial fold.

FIGS. 10A and 10B show a schematic of the contemplated microanatomy of a thread implanted into a patient.

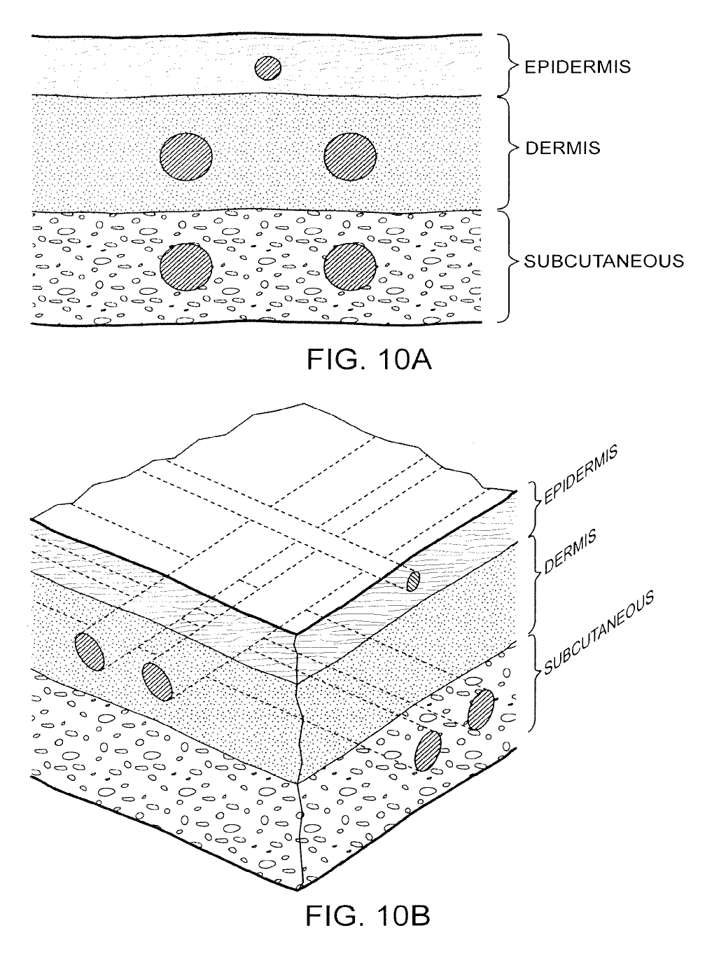

### DETAILED DESCRIPTION

Provided by this disclosure are soft tissue augmentation threads, methods for their preparation and uses thereof and to specific shapes formed there from. However, prior to describing these embodiments in greater detail, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “a thread” includes a plurality of threads.

### 1. DEFINITIONS

Unless defined otherwise, all technical and scientific terms used herein have the same meaning as commonly understood by one of ordinary skill in the art. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The term “soft tissue” refers to tissues that connect, support, or surround other structures and organs of the body, not being bone. Soft tissue includes tendons, ligaments, fascia, skin, fibrous tissues, fat, and synovial membranes, and muscles, nerves and blood vessels which are not connective tissues. In one embodiment, the soft tissue is skin.

As used herein, the term “thread” refers to a long, thin, flexible form of a material. The thread can have a variety of shapes in the cross-section which are discussed below.

The term “biocompatible polymer” refers to polymers which, in the amounts employed, are non-toxic and substantially non-immunogenic when used internally in the patient and which are substantially insoluble in the body fluid of the mammal. Non-limiting examples of biocompatible polymers include one or more of chondroitin sulfate, cyclodextrin, alginate, chitosan, carboxy methyl chitosan, heparin, gellan gum, agarose, cellulose, poly (glycerol-sebacate) elastomer, poly(ethylene glycol)-sebacic acid, poly(sebacic acid-co-ricinoleic acid), guar gum, xanthan gum, and combinations and/or derivatives thereof. Specific combinations include, but are not limited to, collagen/chondroitin sulfate, chitosan/hyaluronic acid and chondroitin sulfate composites, collagen/cyclodextrin polymer (polyβCD), alginate/collagen, alginate cyclodextrin polymer (polyβCD), chitosan/collagen, carboxy methyl chitosan/cyclodextrin polymer (polyβCD), heparin/cyclodextrin polymer (polyβCD), cyclodextin graft/chitosan and cyclodextin graft/alginate. In one embodiment, the polymer is not hyaluronic acid.

The term “hyaluronic acid” or “HA” refers to the polymer having the formula:

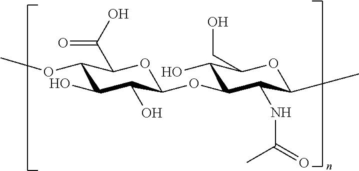

where n is the number of repeating units. All sources of hyaluronic acid are useful, including bacterial and avian sources. Hyaluronic acids used herein have a molecular weight of from about 0.5 MDa (mega Dalton) to about 3.0 MDa. In some embodiments, the molecular weight is from about 0.6 MDa to about 2.6 MDa and in yet another embodiment, the molecular weight is from about 1.4 MDa to about 1.6 MDa.

Chitosan is a linear polysaccharide composed of randomly distributed β-(1-4)-linked D-glucosamine (de-acetylated unit) and N-acetyl-D-glucosamine (acetylated unit). Chitosan is derived from the partial de-acetylation of chitin. Chitosan can be depicted by the formula:

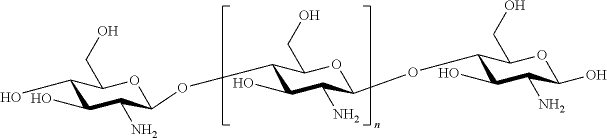

where n is the number of repeating units. Chitosan is the healing substance of chitin. Chitin is the structural element in the exoskeleton of crustaceans (crabs, shrimp, lobster, etc.) and in the cell walls of fungi. Chitosan is highly biocompatible and its unique properties allow it to rapidly clot blood, recently gaining approval in the United States and Europe for use in bandages and other hemostatic agents. Chitin can be depicted by the following formula:

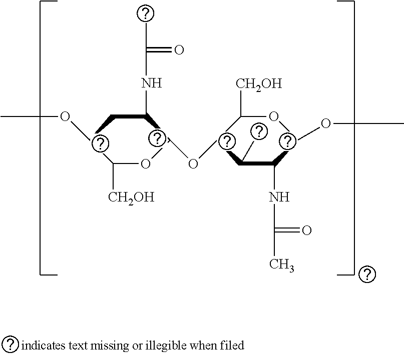

where n is the number of repeating units.

“Alginate” is an anionic polysaccharide distributed widely in the cell walls of brown algae. Alginate includes any salt or ester of alginic acid. In one embodiment, the salt includes sodium, calcium and barium ions.

The term “chondroitin sulfate” refer to a sulfated glycosaminoglycan composed of a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities. Chondroitin sulfate is an important structural component of cartilage and provides much of its resistance to compression.

The term “non-denaturing conditions” refers to conditions which preserve organization of the hyaluronic acid. In some embodiments, non-denaturing conditions include ambient conditions. In another embodiment, non-denaturing conditions includes the use of a desiccant or lyophilization.

The term “ambient conditions” is intended to refer to the typical environmental conditions and preferably, a pressure of about 1 atmosphere and/or temperature of 5 to about 40, and preferably 20 to 30° C.

At least a portion of the thread is cross-linked. The term “cross-linked” is intended to refer to two or more polymer chains which have been covalently bonded via a cross-linking agent. Such cross-linking is differentiated from intermolecular or intramolecular dehydration which results in lactone or anhydride formation within a single polymer chain or between two or more chains. Although, it is contemplated that intramolecular cross-linking may also occur in the threads.

“Cross-linking agents” contain at least two reactive groups that create covalent bonds between two or more molecules. The cross-linking agents can be homobifunctional (i.e. have two reactive ends that are identical) or heterobifunctional (i.e. have two different reactive ends). The cross-linking agents to be used should comprise complimentary functional groups to that of biocompatible polymer such that the cross-linking reaction can proceed. Suitable cross-linking agents include, by way of example only, butanediol diglycidyl ether (BDDE), divinyl sulfone (DVS), and 1-ethyl-3-(3-dimethylaminopropyl) carbodimide hydrochloride (EDC), or a combination thereof. In one embodiment, the cross-linking agent is BDDE.

The term “ultimate tensile strength” is intended to refer to the tensile strength of the thread which has been normalized with respect to cross-sectional area. The term “tensile strength” is intended to refer to the maximum load a thread can withstand without failing when subjected to tension. In one embodiment, it is contemplated that the ultimate tensile strength is sufficient to pull the thread through the skin and manipulate it once in the skin such that the integrity of the thread is not substantially compromised by, for example, breaking or segmenting. It is contemplated that threads preferably have an ultimate tensile strength of about 3 kpsi (“kilopounds per square inch”) or greater, or 5 kpsi or greater, or 10 kpsi or greater, or 15 kpsi or greater or 20 kpsi or greater or 50 kpsi or greater or 75 kpsi or greater.

The threads can be made into a variety of shapes. The term “substantially cylindrical” refers to a thread wherein the cross-section of the thread is round. The term “substantially” as used to refer to shapes of the threads means that at least 50% of the thread has the shaped described. The term substantially is also used to encompass threads which have a variety shapes along the length of the thread. For example, a thread could be substantially cylindrical but the ends of the thread may be tapered. The substantially cylindrical threads can be provided when the contact angle of the gel composition and the substrate on which it is extruded have an equilibrium contact angle of greater than about 90 degrees.

The term “substantially D-shaped” refers to a thread wherein the cross-section is D-shaped or substantially semi-circular. The substantially D-shaped threads have one flat side and one substantially round side. The substantially D-shaped threads can be provided when the contact angle of the gel composition and the substrate on which it is extruded have an equilibrium contact angle of about 90 degrees.

The term “substantially ribbon-shaped” refers to a thread wherein the thickness of the thread is less than about 50% of the width of the thread. In some embodiments, the cross-section is substantially rectangular. The ribbon-shaped threads can be provided when the contact angle of the gel composition and the substrate on which it is extruded have an equilibrium contact angle of less than about 90 degrees. Alternatively, the ribbon-shaped threads can be formed by cutting a wet gel to achieve the desired cross-sectional shape. “Ribbon-shaped” may also include shapes that are substantially ellipsoidal. The term “substantially ellipsoidal” refers to a thread wherein the cross-section is substantially oblong or elliptical.

The term “non-peptidic” refers to a material, examples of which are described herein, wherein at least a portion of which is not composed substantially of peptides and/or proteins. It is understood that the presence of amino acid residues attached to the polymer scaffolds disclosed herein do not render such scaffolds peptidic so long as the total molecular weight of the polymer is attributed to about 10% or less of amino acids. In one embodiment, the non-peptidic materials described herein contain no amino acid residues derived from one of the 20 naturally occurring amino acids.

The term “self-swellable” or “self-expandable” refers to materials that are capable of swelling or expanding in size when in contact with an aqueous environment, such as for example, when placed in the human body. In one embodiment, the threads are self-swellable between about 20% and 1000%.

The term “percent moisture” is intended to refer to the total percent of water by weight. In one embodiment, the percent moisture is about 30% or less, or alternatively, about 15% or less, or alternatively, about 10% or less. This can typically be measured by Karl Fisher titration.

The term “non-compressible” refers to a material that does not compress more than about 20% when a force is applied. In some cases, the material cracks, breaks, or otherwise loses structural integrity rather than compresses by greater than 20%.

The term “carbohydrate based” refers to a material that is based on a sugar or polysaccharide. Examples include chitosan and alginates.

The term “controlled swellability” refers to the relative percentage that the biocompatible polymer swells when contacted with a moisture source (i.e., water, buffer, bodily fluid, etc.). This can be controlled by various means, such as the weight percent biocompatible polymer in the gel solution, the nature of the polymer/polymer composition, the presence, nature and/or amount of a cross-linking agent used, thickness of the thread, etc.

The term “therapeutic agent” can include one or more therapeutic agents. In still other of the above embodiments, the therapeutic agent is an anesthetic, including but not limited to, lidocaine, xylocaine, novocaine, benzocaine, prilocaine, ripivacaine, propofol or combinations thereof. In still other of the above embodiments, the therapeutic agent includes, but is not limited to, epinephrine, ephedrine, aminophylline, theophylline or combinations thereof. In still other of the above embodiments, the therapeutic agent is botulism toxin. In still other of the above embodiments, the therapeutic agent is laminin-511. In still other of the above embodiments, the therapeutic agent is glucosamine, which can be used, for example, in the treatment of regenerative joint disease. In still other of the above embodiments, the therapeutic agent is an antioxidant, including but not limited to, vitamin E or all-trans retinoic acid such as retinol. In still other of the above embodiments, the therapeutic agent includes stem cells. In still other of the above embodiments, the therapeutic agent is insulin, a growth factor such as, for example, NGF (nerve growth factor), BDNF (brain-derived neurotrophic factor), PDGF (platelet-derived growth factor) or Purmorphamine Deferoxamine NGF (nerve growth factor), dexamethasone, ascorbic acid, 5-azacytidine, 4,6-disubstituted pyrrolopyrimidine, cardiogenols, cDNA, DNA, RNAi, BMP-4 (bone morphogenetic protein-4), BMP-2 (bone morphogenetic protein-2), an antibiotic agent such as, for example, B lactams, quinolones including fluoroquinolones, aminoglycosides or macrolides, an anti-fibrotic agent, including but not limited to, hepatocyte growth factor or Pirfenidone, an anti-scarring agent, such as, for example, anti-TGF-b2 monoclonal antibody (rhAnti-TGF-b2 mAb), a peptide such as, for example, GHK copper binding peptide, a tissue regeneration agent, a steroid, fibronectin, a cytokine, an analgesic such as, for example, Tapentadol HCl, opiates, (e.g., morphine, codone, oxycodone, etc.) an antiseptic, alpha-beta or gamma-interferon, EPO, glucagons, calcitonin, heparin, interleukin-1, interleukin-2, filgrastim, a protein, HGH, luteinizing hormone, atrial natriuretic factor, Factor VIII, Factor IX, or a follicle-stimulating hormone.

The term “diagnostic agent” refers to an agent which is used as part of a diagnostic test (e.g., a fluorescent dye to be used for viewing the thread in vivo). In one embodiment, the diagnostic agent is soluble TB (tuberculosis) protein.

The term “lubricity-enhancing agent” is intended to refer to a substance or solution which when contacted with the dry thread, acts to lubricate the dry thread. A lubricity-enhancing agent can comprise, for example, water and/or an alcohol, an aqueous buffer, and may further comprise additional agents such as polyethylene glycol, hyaluronic acid, and/or collagen.

The term “biodegradation impeding agent” is intended to refer to a biocompatible substance that slows or prevents the in vivo degradation of the thread. For example, a biodegradation impeding agent can include hydrophobic agents (e.g., lipids) or sacrificial biodegradation agents (e.g., sugars).

The term “failure load” is intended to refer to the maximum weight which, when applied to the thread, causes the thread to fail. By “failing,” it meant that the thread can break or segment or otherwise lose structural integrity. In some embodiments, the failure stress is about 0.1 pounds or 0.22 kilograms or greater.

The term “aqueous gel composition” or “gel composition” or “gel mixture” is intended to refer to an aqueous composition comprising water, biocompatible polymer, and a cross-linking agent. In some embodiments, the composition may further comprise a buffer such that that the pH of the solution changes very little with the addition of components of the composition. In these embodiments, the composition is referred to as an aqueous buffered gel composition. The pH of the buffered gel composition is typically about 7. In some embodiments, the aqueous gel buffered composition comprises phosphate buffered saline. In some embodiments, additional solutes are added to adjust the osmolarity and ion concentrations, such as sodium chloride, calcium chloride, and/or potassium chloride.

The term “buffer” is intended to refer to a solution comprising a mixture of a weak acid and its conjugate base or a weak base and its conjugate acid. Buffer solutions include, but are not limited to, 2-amino-2-methyl-1,3-propanediol, 2-amino-2-methyl-1-propanol, L-(+)-tartaric acid, D-(−)-tartaric acid, ACES, ADA, acetic acid, ammonium acetate, ammonium bicarbonate, ammonium citrate, ammonium formate, ammonium oxalate, ammonium phosphate, ammonium sodium phosphate, ammonium sulfate, ammonium tartrate, BES, BICINE, BIS-TRIS, bicarbonate, boric acid, CAPS, CHES, calcium acetate, calcium carbonate, calcium citrate, citrate, citric acid, diethanolamine, EPP, ethylenediaminetetraacetic acid disodium salt, formic acid solution, Gly-Gly-Gly, Gly-Gly, glycine, HEPES, imidazole, lithium acetate, lithium citrate, MES, MOPS, magnesium acetate, magnesium citrate, magnesium formate, magnesium phosphate, oxalic acid, PIPES, phosphate buffered saline, piperazine potassium D-tartrate, potassium acetate, potassium bicarbonate, potassium carbonate, potassium chloride, potassium citrate, potassium formate, potassium oxalate, potassium phosphate, potassium phthalate, potassium sodium tartrate, potassium tetraborate, potassium tetraoxalate dehydrate, propionic acid solution, STE buffer solution, sodium 5,5-diethylbarbiturate, sodium acetate, sodium bicarbonate, sodium bitartrate monohydrate, sodium carbonate, sodium citrate, sodium chloride, sodium formate, sodium oxalate, sodium phosphate, sodium pyrophosphate, sodium tartrate, sodium tetraborate, TAPS, TES, TNT, TRIS-glycine, TRIS-acetate, TRIS buffered saline, TRIS-HCl, TRIS phosphate-EDTA, tricine, triethanolamine, triethylamine, triethylammonium acetate, triethylammonium phosphate, trimethylammonium acetate, trimethylammonium phosphate, Trizma® acetate, Trizma® base, Trizma® carbonate, Trizma® hydrochloride or Trizma® maleate.

The term “aqueous solvent” is intended to refer to a non-toxic, non-immunogenic aqueous composition. The aqueous solvent can be water and/or an alcohol, and may further comprise buffers, salts and other such non-reactive solutes.

The term “contact angle” or “equilibrium contact angle” refers to a measure of a liquid's affinity for a solid and quantifies the degree of a liquid drop's spread when placed on the solid. In one embodiment, the liquid is the aqueous gel composition and the rigid or solid surface is the substrate on which the composition is extruded. The contact angle is a measure of the angle that the edge of an ideal drop makes with a flat surface. The lower the contact angle is, the greater the attraction between the surface and the liquid. For example, water spreads almost completely on glass and has a very low contact angle of nearly 0 degrees. Mercury, in contrast, beads up and spreads very little; its contact angle is very large.

### 2. SOFT TISSUE AUGMENTATION THREAD

In some embodiments, the present disclosure is directed to a soft tissue augmentation thread comprising a biocompatible polymer wherein at least a portion of which is non-peptidic, self-swellable or self-expandable, and carbohydrate based.

In some embodiments, the present disclosure is directed to a soft tissue augmentation thread comprising a biocompatible polymer having an elastic modulus and wherein upon delivery to skin of a patient, the polymer decreases or increases its modulus.

The elastic modulus can be any elastic modulus, such as Young's modulus (stretch), shear modulus and/or bulk modulus (3-dimensional expansion).

Exemplary biocompatible polymers include chondroitin sulfate, cyclodextrin, alginate, chitosan, carboxy methyl chitosan, heparin, gellan gum, agarose, cellulose, guar gum, xanthan gum, and combinations and/or derivatives thereof. In one embodiment, the biocompatible polymer is not hyaluronic acid. In one embodiment, the biocompatible polymer is not collagen.

In one embodiment, the biocompatible polymer comprises one or more of chondroitin sulfate, cyclodextrin, alginate, chitosan, carboxy methyl chitosan, heparin, gellan gum, agarose, cellulose, poly (glycerol-sebacate) elastomer, poly(ethylene glycol)-sebacic acid, poly(sebacic acid-co-ricinoleic acid), guar gum, xanthan gum, and combinations and/or derivatives thereof.

The thread is formed by drying an aqueous gel composition which comprises a biocompatible polymer, and optionally a cross-linking agent, under non-denaturing conditions and preferably ambient conditions.

In some embodiments, at least a portion of the thread is cross-linked.

The physical properties of the thread can be tailored for a specific use by adjusting the components in the aqueous gel composition and adjusting the method of producing the thread as discussed below.

The half-life of the soft tissue augmentation thread in vivo can be controlled by controlling the thickness of the thread, the density, the molecular weight of the biocompatible polymer, the amount of cross-linking, and the degree of moisture (e.g., swellability), which can then be further controlled by adjusting the amounts of biocompatible polymer and optionally a cross-linking agent both individually and relatively. It is contemplated that the soft tissue augmentation threads disclosed herein can have a half-life in vivo of from about 1 month to up to about 12 months.

The percent swell of soft tissue augmentation thread can range from about 1% to greater than about 1000% based on the total weight. The swellability (or percent swell) of the thread can be controlled by adjusting the percent biocompatible polymer in the gel and/or controlling the amount and type of cross-linking agent added. It is contemplated that a lower percent moisture thread would result in a thread with a higher tensile strength. In some embodiments, the thread has no more than about 30% percent, or no more than 15%, or no more than 10% by weight moisture based on the total weight. The percent moisture will be determined by the environment to which the thread is subjected to during or after the drying process.

As mentioned above, in some embodiments, at least a portion of the biocompatible polymer is cross-linked. The cross-linking agent to be used should comprise complimentary functional groups to that of biocompatible polymer such that the cross-linking reaction can proceed. The cross-linking agent can be homobifunctional or heterobifunctional. It is contemplated that the percent moisture of the thread may be at least partially controlled by the type of cross-linking agent employed. For example, if the cross-linking leaves the carboxyl groups of the biocompatible polymer unfunctionalized, the percent moisture of the thread may higher than functionalized biocompatible polymers. Suitable cross-linking agents include, but are not limited to, butanediol diglycidyl ether (BDDE), divinyl sulfone (DVS), and 1-ethyl-3-(3-dimethylaminopropyl) carbodimide hydrochloride (EDC), or a combination thereof. In one embodiment, the cross-linking agent is BDDE.

The amount of cross-linking agent, or cross-link density, should be high enough such that the thread formed thereby is elastomeric, however it should not be so high that the resulting thread is too rigid so it cannot be moved within the skin during delivery when used as a soft tissue augmentation product. The appropriate stiffness or elastic modulus is determined by the intended use of the thread. It is contemplated that the degree of cross-linking may be determined so as to provide the improved mechanical properties of increased strength and/or an enhanced ability to promote fibrogenesis.

It is contemplated that the amount of cross-linker in the gel formulation used to make the thread can be between about 0.1% and about 5% by volume. In other embodiments, the amount of cross-linker is from about 0.2% to about 2% or from about 0.2% to about 0.8% by volume. However, the amount may vary depending on the use and composition of the thread. It is contemplated that the thread is cross-linked throughout the length of the thread. In some embodiments, it is contemplated that the cross-linking is substantially uniform throughout the length of the thread.

### 3. METHODS OF MAKING THE THREADS

The disclosure is also directed to a method of making the thread. The method comprises drying under non-denaturing and preferably ambient conditions an aqueous gel composition comprising a biocompatible polymer wherein at least a portion of which is non-peptidic, self-swellable or self-expandable, non-compressible, and carbohydrate based, and optionally a cross-linking agent, to provide a dry thread.

Typically, the aqueous gel composition comprises water and can optionally comprise phosphate buffered saline (PBS) and optionally have a pH of about 7. To the water or PBS, is added the desired amount of biocompatible polymer, which is from about 1% to about 30% by weight, and is preferably about 5 to about 10% by weight. The relative amount of biocompatible polymer can be adjusted based on its molecular weight to provide a composition of desired viscosity. After adding the biocompatible polymer, it is allowed to dissolve slowly to form a gel. The viscosity of the gel can be determined by methods known in the art. Once the gel is formed, from about 0.1% to about 2.0% by volume of cross-linking agent is optionally added and the gel solution mechanically stirred. The cross-linking agent in some embodiments is BDDE and the amount used is from about 0.2% to about 1.0% by volume.

In some embodiments, the gel composition is degassed prior to extrusion to minimize air bubbles after extrusion. The degassing can be done by applying a standard vacuum pump. If desired, the gel can be degassed using a standard freeze-pump-thaw procedure which is known by one of skill in the art. Air bubbles can reduce the structural integrity of the thread by causing weak spots.

To form the thread, the gel composition is typically extruded onto a substrate which is more thoroughly discussed in Example 1 to form a wet thread. The composition is extruded using a pressurized syringe affixed to a nozzle. The nozzle can have various geometries, such as various lengths, internal diameters and shapes. The nozzle may be circular or non-circular in shape, for example, a flattened shape or a “D” shape. The syringe nozzle may be anywhere from about a 15 gauge and a 25 gauge syringe nozzle. Typically, the pressure employed is from about 10 to about 2000 psi or from about 20 to about 240 psi. The pressure requirements are dictated by the nozzle geometry. The pressure can be applied pneumatically, for example using ambient air or nitrogen, hydraulically, or mechanically. The speed at which the gel is extruded is selected so as to minimize breakage in the length of the thread and maximize a consistent shape.

Various substrates are contemplated for use by methods described herein. Substrates include hydrophilic and hydrophobic substrates and may be selected from, but are not limited to, polytetrafluoroethylene (PTFE), expanded PTFE, nylon, polyethylene terephthalate (PET), polystyrene, silicon, polyurethane, and activated cellulose.

The substrate employed, along with the viscosity of the gel composition, dictates the general shape of the thread. For example, if the gel and the substrate have an equilibrium contact angle of less than 90 degrees, it is contemplated that the thread formed will be substantially ribbon-shaped. Further, if the gel and the substrate have an equilibrium contact angle of about 90 degrees, the thread formed will be substantially D-shaped. Still further, if the gel and the substrate have an equilibrium contact angle of greater than 90 degrees, then the thread formed will be substantially round. For example, a 10% 1.5 MDa gel will have a substantially circular cross-section (e.g., about 80% of a circle) when extruded on PTFE, while a 5% 1.5 MDa gel will form a flat ribbon when extruded on PTFE.

Alternative to pressurized extrusion, the gel composition can be rolled out into an elongated cylinder and/or cut into elongated strips before drying.

It is contemplated that the threads can be sterilized using typical sterilization methods known in the art, such as autoclave, ethyleneoxide, gamma irradiation, steam, electron beam (e-beam), supercritical CO₂ (with peroxide), freeze-drying, etc. For example, the threads can be sterilized using electron beam (e-beam) sterilization methods.

The wet thread is then dried to form a dry thread. The drying step is required to form threads with a sufficient tensile strength, as discussed below. As the thread may lose some of its organization properties when exposed to heat in excess of water boiling temperature, it is preferred that the drying step be performed under ambient conditions. It is contemplated that by drying under ambient conditions, the biocompatible polymer is allowed to organize. In embodiments where a cross-linking agent is added, it is contemplated that the biocompatible polymer is allowed to organize as the cross-linking reaction is taking place or before it takes place. This drying procedure provides a thread with a higher tensile strength, such as, for example, an ultimate tensile strength of the dry thread of greater than about 5 kpsi, or greater than about 10 kpsi, or greater than about 15 kpsi, or greater than about 20 kpsi. In addition, the threads have a failure stress of greater than about 0.5 pounds, or greater than about 0.6 pounds, or greater than about 0.7 pounds, or greater than about 0.8 pounds, or greater than about 0.9 pounds, or greater than about 1 pound.

The thread is allowed to dry for anywhere from about 30 minutes to about 72 hours to form threads having a diameter of from 0.05 mm to about 1.0 mm and having no more than 30% by weight moisture. In some embodiments, the thread can be dried for about 12 hours or about 24 hours. It is contemplated that the larger the molecular weight of the biocompatible polymer employed or the more concentrated the biocompatible polymer in the composition, the longer the drying times that are required. Further, in gels comprising a cross-linking agent, during the drying process, a non-thermal stimulus, such UV light, radiation, or a chemical initiator, may be employed to assist in the cross-linking reaction.

In some embodiments, after drying, the thread is washed with an aqueous solvent, a gas or a supercritical fluid. In some embodiments, this washing removes excess cross-linking agent. The washing can be accomplished by a variety of methods, such as submersion in an aqueous solvent or by using a concurrent flow system by placing the thread in a trough at an incline and allowing an aqueous solvent to flow over the thread. Threads can also be suspended, for example vertically, and washed by dripping or flowing water down the length of the thread.

In one embodiment, water is used to wash the threads. In this embodiment, the water not only washes the threads to remove excess cross-linking agent, it also rehydrates the thread into a hydrated elastomeric state. Optionally and as necessary, the thread is mechanically stretched while hydrated, either soon after being hydrated or gradually before the first drying or after the rehydrating. The stretching or absence of stretching can provide a thread of the desired length and/or rehydration swelling volume. In some embodiments, the length of the thread can be from about 0.5 cm to about 15 cm.

After the thread is rehydrated it is allowed to dry again under ambient conditions for from anywhere from 30 minutes to about 72 hours. Upon drying, the thread, in some embodiments, cures to provide a more uniform surface of the thread.

This washing hydration/dehydration step can be performed multiple times to allow excess unreacted reagent to be washed from the thread or to continue to improve the degree of cross-linking. Additional washing with organic solvents, such as ethanol, may also be used.

### 4. MODIFICATION OF THREADS

In addition to washing the thread, it can also be further functionalized by adsorbing a sufficient amount of a member selected from the group consisting of a therapeutic agent, a diagnostic agent, a fibrogenesis-enhancing agent, a biodegradation impeding agent, a lubricity-enhancing agent and combinations thereof, optionally followed by re-drying the thread. Such therapeutic agents include antibacterials, anesthetics, dyes for viewing placement in vivo, and the like. In some embodiments, a dry or hydrated thread is coated to alter the properties with a bioabsorbable biopolymer as described herein. In some embodiments, the polymer is collagen, PEG, PLGA or a phase transfer Pluronic™ which can be introduced as a liquid and which solidifies in vivo.

In one embodiment, the thread can be coated such that the rate at which the thread is rehydrated. For example, the thread can be coated with a hydrophobic layer, such as a lipid. The thickness of the lipid layer can then be adjusted to achieve the desired rate of rehydration. In another embodiment, the thread can be coated with an aqueous composition of hyaluronic acid. In another embodiment, the thread can be coated with an aqueous composition of collagen. This can be performed just prior to implantation of the thread to act as a lubricant. It is also contemplated that this coating may slow the rate of hydration of the thread. In some embodiments, the thread is coated, either totally or in part, with the gel composition to form a layered material. For woven constructs, whether single layer or 3D, can be coated in their entirety to create weaves or meshes with altered physical properties from that of a free-woven mesh.

The threads as disclosed herein can be braided, coiled, layered or woven. In some embodiments, braids may be formed from the threads described above. A braid can be formed by intertwining three or more threads wherein each thread is functionally equivalent in zigzagging forward through the overlapping mass of the others. The braids can be a flat, three-strand structure, or more complex braids can be constructed from an arbitrary (but usually odd) number of threads to create a wider range of structures, such as wider ribbon-like bands, hollow or solid cylindrical cords, or broad mats which resemble a rudimentary perpendicular weave.

In one embodiment, a plasticizer is added to adjust the stiffness of the thread. Alternatively, or in addition to, threads of varying stiffness may be weaved together to produce a braided thread or material having the desired stiffness.

In some embodiments, a three-dimensional structure may be constructed by weaving or wrapping or coiling or layering the threads described above. In other embodiments, a three-dimensional structure may be constructed by weaving or wrapping or coiling or layering the braids described above. In still other embodiments, a three-dimensional structure may be constructed by weaving or wrapping or coiling or layering the cords described above. In still other embodiments, a three-dimensional structure may be constructed by weaving or wrapping or coiling or layering the meshes described above.

In some embodiments, a three-dimensional, cylindrical implant is made of any of the threads is provided. An exemplary use for such an implant is for nipple reconstruction. In some embodiments, the threads used to make the cylindrical implant are cross-linked and include chrondrocyte adhesion compounds. In other embodiments, the cylindrical shape is provided by multiple, concentric coils of threads.

### 5. METHODS OF USING THE SOFT TISSUE AUGMENTATION THREADS

The threads, braids, cords, woven meshes or three-dimensional structures described herein can be used, for example, to fill wrinkles, to fill aneurysms, occlude blood flow to tumors, (i.e., tumor occlusion), in eye-lid surgery, in penile augmentation (e.g., for enlargement or for sensitivity reduction, i.e., pre-mature ejaculation treatment), inter-nasal (blood-brain barrier) delivery devices for diagnostic and/or therapeutic agents, corneal implants for drug delivery, nose augmentation or reconstruction, lip augmentation or reconstruction, facial augmentation or reconstruction, ear lobe augmentation or reconstruction, spinal implants (e.g., to support a bulging disc), root canal filler (medicated with therapeutic agent), glottal insufficiency, laser photo-refractive therapy (e.g., thread/weave used as a cushion), scaffolding for organ regrowth, spinal cord treatment (BDNF and NGF), in Parkinson's disease (stereotactic delivery), precise delivery of therapeutic or diagnostic molecules, in pulp implantation, replacement pulp root canal treatment, shaped root canal system, negative pressure wound therapy, adhesion barriers and wound dressings.

### Methods of Treating a Wrinkle

It is contemplated that threads have an improved ability to promote regeneration and/or tissue repair in vivo by forming a scaffold-like structure in the body for collagen deposition. This tissue repair could prolong the “filler” effects of the thread when used to treat or fill a wrinkle in vivo far beyond the half-life of the unmodified soft tissue augmentation thread. This is described in Example 7.

In some embodiments, the present disclosure is directed to a method of treating a wrinkle in a patient in need thereof by 1) inserting the thread into the skin of the patient adjacent to or under the wrinkle; and 2) applying the thread adjacent to or under the wrinkle thereby treating the wrinkle. These steps can be performed at least once and up to 6 times to treat each wrinkle. In some embodiments, the thread is attached to the proximal end of a needle The thread is inserted by a needle which needle is then removed. Optionally and as necessary, the thread is hydrated with water or saline, or by the fluids normally perfusing the surrounding tissue. Further, the remainder of the wrinkle can be filled with a biocompatible material such as a phase transfer Pluronic™ which can be introduced as a liquid and which solidifies in vivo. Alternatively, conventional soft tissue augmentation products (i.e., Restylane®, Juvaderm®, etc.) can be introduced to fill the wrinkle. In either case, the formed web acts to maintain the biocompatible filler at the site of the wrinkle.

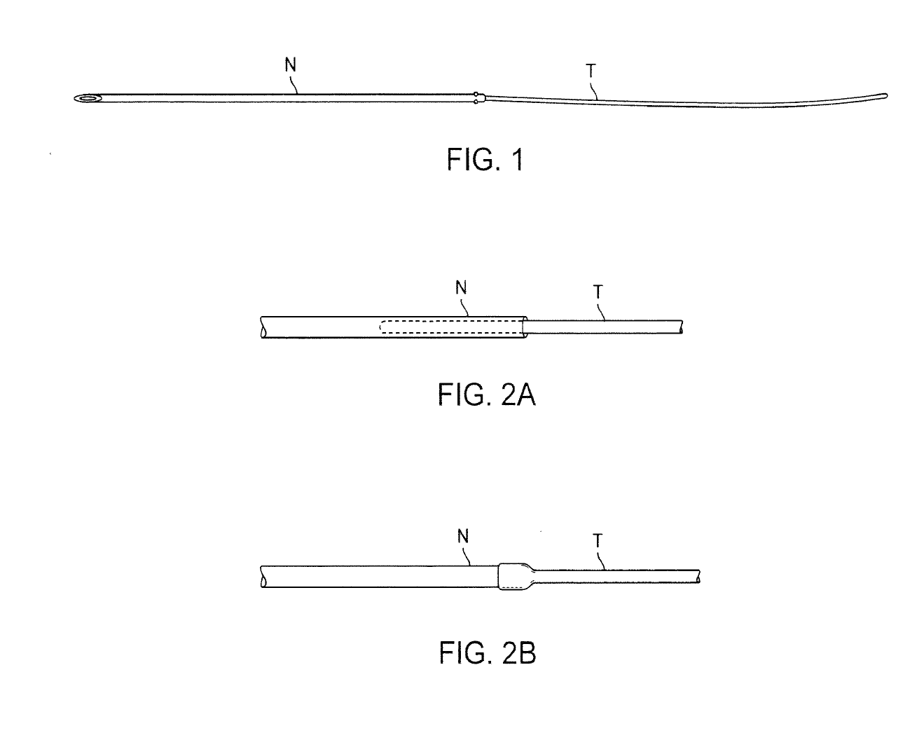

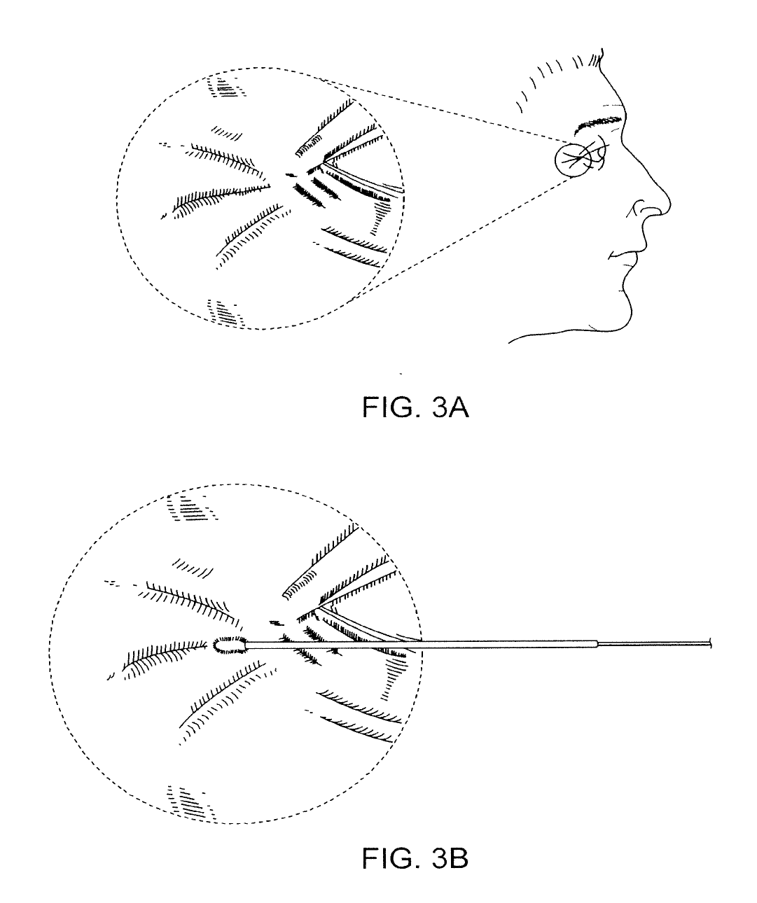

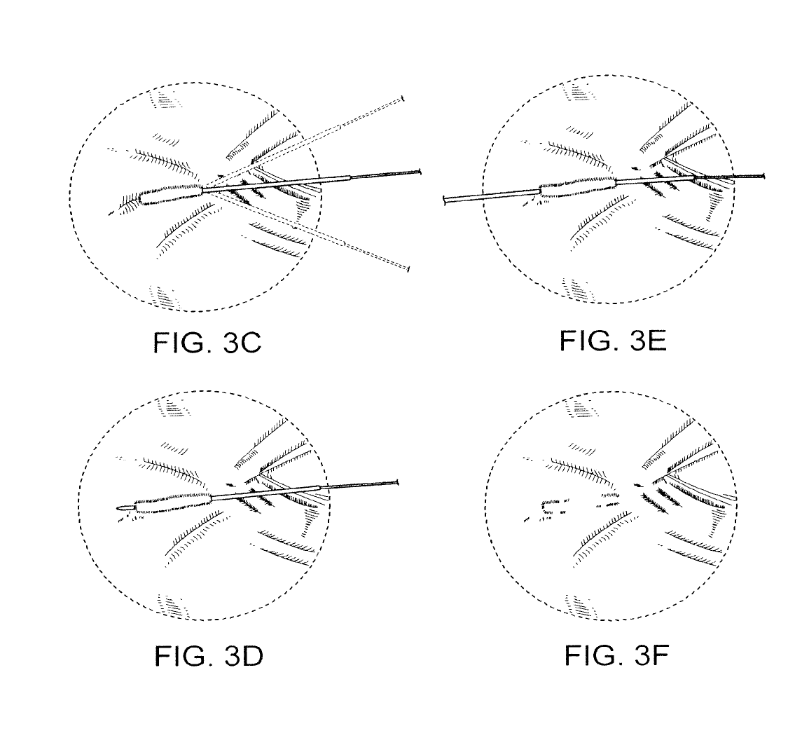

In some embodiments, a method of treating a wrinkle in a subject is provided. In some embodiments, the attending clinician may numb the treatment area according to procedures known in the art using a variety of anesthetics, including, but not limited to, topical lidocaine, ice or a block with lidocaine injection. For example, the wrinkle may be in the peri-orbital region as illustrated in FIG. 3A. The thread may be attached to a needle as illustrated, for example, in FIGS. 1, 2A and 2B. The distal end of the needle may be inserted through the skin surface of the subject into the skin adjacent to or within the wrinkle as illustrated, for example, in FIG. 3B. In some embodiments, the thread is inserted into the subcutaneous space instead of the dermis. The needle then may traverse the skin of the subject beneath the wrinkle as illustrated, for example, in FIG. 3C. The needle then may exit the skin of the subject at the opposite margin of the wrinkle, as illustrated, for example, in FIG. 3D. The needle may then be pulled distally until it is removed from the subject such that the thread is pulled into the location previously occupied by the needle beneath the wrinkle, as illustrated, for example, in FIG. 3E. Finally, excess thread is cut from the needle at the skin surface of the subject which leaves the thread implanted as illustrated, for example, in FIG. 3F.

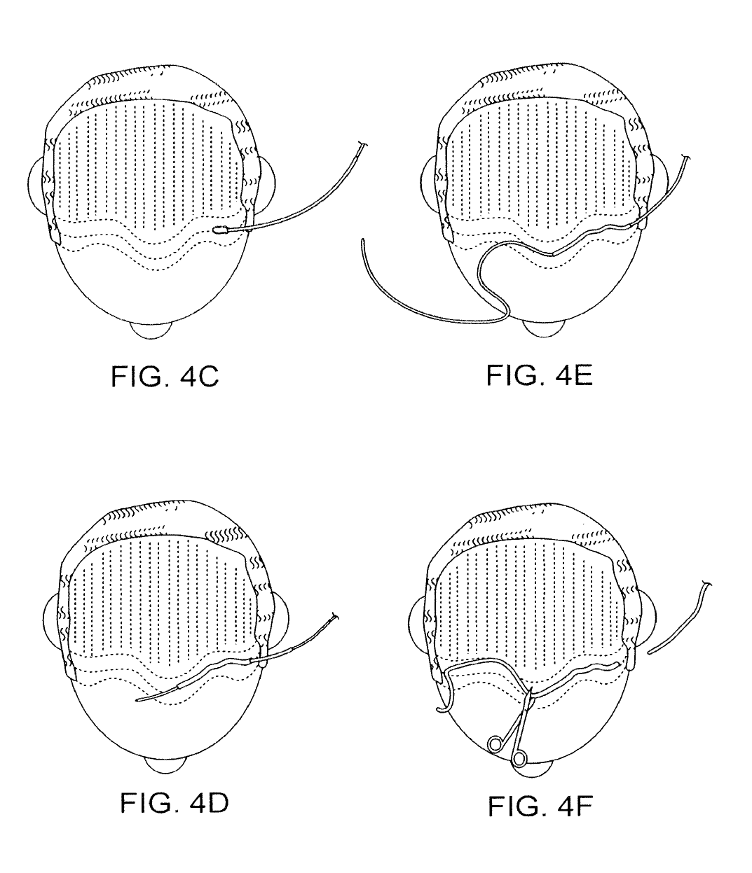

While not wishing to be bound by theory, the method above may successfully treat wrinkles as shown in FIGS. 5A, 5B and 5C. A typical wrinkle is illustrated in FIG. 5A. FIG. 5B illustrates a thread implanted beneath a wrinkle that is not yet hydrated. As the thread implanted beneath the wrinkle becomes fully hydrated the surface appearance of the wrinkle is concurrently flattened as illustrated in FIG. 5C.

In some embodiments, the thread is manipulated in such a fashion such that one end of the thread is sufficiently hard such that the thread is used to penetrate the skin. This may be accomplished by coating the thread with a hardening material, such as a sugar coating, In another embodiment, the thread is coated in its entirety, for example with a sugar coating, to provide the thread with increased columnar strength.

### Facial Contouring

It is contemplated that the threads are useful in facial contouring. What is meant by facial contouring is that the threads can be applied to any area of the face, neck, or chest that the patient desires to have augmented, including, by way of example only, the lips, the nasolabial fold, and tear trough.

Lip augmentation is a commonly desired aesthetic procedure. Typically, the aesthetic goal is fuller, plumper lips. Some psychology studies have described an increased attraction by males for females with fuller lips (Lip Size Key to Sexual Attraction, 4 Mar. 2003. http://news.bbc.co.uk/2/hi/health/2817795.stm). The hypothetical explanation for this phenomenon is that lip fullness or plumpness is correlated with increased estrogen levels and is therefore perceived as a sign of fertility. Available treatment options for lip augmentation include gels and surgical procedures. Areas of enhancement can include the vermillion border (or white roll) for lip effacement and contouring and the wet-dry mucosal junction for increasing fullness. Other techniques include more diffuse infiltration of the orbicularis oris muscle.

Lip contouring and augmentation by temporary soft tissue augmentation products is a popular, low risk option due to the minimal invasiveness and temporary nature of the procedure. The major shortcomings of soft tissue augmentation products currently used in lip procedures are that it is (a) painful, (b) difficult to consistently and homogenously inject the gel into the desired location, and (c) the gel can migrate over the lifetime of the implant causing the aesthetic results to change.

The present disclosure addresses the shortcomings described above. Beyond addressing the above-listed shortcomings for existing temporary soft tissue augmentation products described above, it has been found that the thread-based method of enhancing lip appearance is very quick. A typical patient may have 3 threads in their lip(s) in only 3 minutes. Current soft tissue augmentation product lip procedures can take 15 to 20 minutes.

In embodiments, directed to facial contouring, the attending clinician may numb the treatment area according to procedures known in the art using a variety of anesthetics, including, but not limited to, topical lidocaine, ice or a block with lidocaine injection. Threads can be attached to the proximal end of a needle and pulled into the lip. The needle can serve as a precise guide, and also be used to predict and correct the implant location prior to pulling the thread into the desired location. This precise delivery mechanism can be used to deliver threads along the vermillion border for contouring, superficially if desired, as well as at the wet-dry junction for plumping, deeper into the lip if desired.

It is contemplated that when the thread is used for facial contouring, any number of threads may be used depending on the desired effect and the size of the thread. For example, description of the procedure done for the lip augmentation and contouring is discussed herein.

It is has been surprisingly and unexpectedly found that that threads may be implanted in various tissue planes of the patient to provide a more natural look when performing facial contouring. For example, the threads may be implanted in a manner that forms a hammock in the desired location. Given the unique properties of the threads, the attending clinician may deposit or implant the threads in the epidermis, the dermis, and the subcutaneous layer. This technique is referred to as stratifying the thread implantation.

This technique is enabled by the precision with which the threads can be placed, and their size relative to the skin and underlying structures. Threads can impart different effects on facial features such as wrinkles, contours, folds and troughs depending on where they are implanted.

For example, recent clinical experience indicates that placing a thread (in this case one that was approximately 0.008″ in diameter) deeply, for example in the subcutaneous space, along the axis of a forehead wrinkle can help soften then appearance of the wrinkle that forms when the patient animates, by flexing their forehead, which would typically exacerbate the appearance of the wrinkle. These types of dynamic wrinkles are currently only well treated with Botox®, which has the undesirable effect of preventing the patient from expressing all facial expressions. Further, recent clinical experience shows that static wrinkles, ones that are visible in repose, can be effectively treated by placement of a thread (from 0.004″ to 0.008″ in diameter) superficially, for example within the skin.

The technique of stratifying the thread implant in various tissue planes is also successfully used in improving the appearance of nasolabial folds (up to four 0.008″ threads), glabellar lines, marionette lines, and lips.

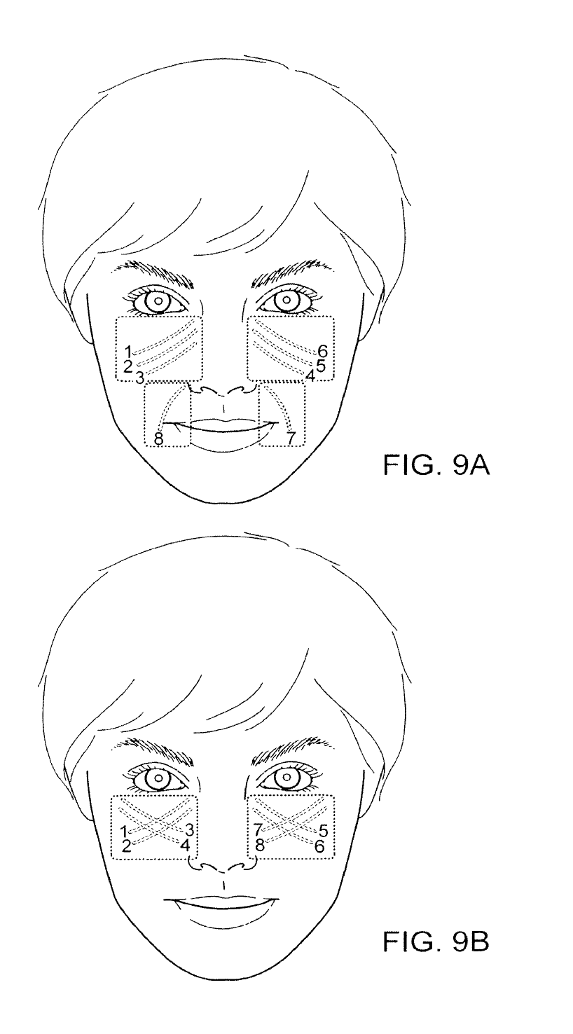

This is another technique that is enabled by the threads and their implantation method. To smooth the appearance of hollows or troughs such as the tear trough, or otherwise contour the face in areas such as the cheek bones, chin, for example, threads can be implanted in hatch (see, FIG. 9A) and or cross-hatched patterns (see, FIG. 9B) to effect areas greater than the width of a single thread. As seen in FIGS. 9A and 9B, two patients have their tear troughs effectively smoothed out by placing threads parallel in one case (FIG. 9A) and cross-hatched in another case (FIG. 9B). The cross-hatching could be done obliquely to the initial direction, as was the case in FIG. 9B, or perpendicularly. Further, the hatches can be at different tissue planes.

In another embodiment of this technique, the hatching can be done obliquely to the directionality of the area being treated. For example, in FIG. 9A below the threads are placed aligned to the axis of the tear trough. Instead, the threads could be placed obliquely to the axis of the tear trough to support the tissue in the area differently.

It is contemplated that implanting the threads in various planes may also be done in the treatment of wrinkles as described above.

### Wound Therapy

In some embodiments, the threads, braids, cords, woven meshes or three-dimensional structures described herein are used in wound dressings including negative pressure wound dressings.

In some embodiments, wound dressing remains in contact with the wound for at least 72 hours. In other embodiments, the negative pressure wound dressing remains in contact with the wound for at least 1 week. In still other embodiments, the wound dressing remains in contact with the wound for at least 2 weeks. In still other embodiments, the wound dressing remains in contact with the wound for at least 3 weeks. In still other embodiments, the wound dressing remains in contact with the wound for at least 4 weeks. In the above embodiments, it should be understood that granulation tissue is not retaining the threads, braids, cords, woven meshes or three-dimensional structures described herein as these components are fully absorbable. In some of these embodiments, the wound dressing is between about 1 cm and about 5 cm thick. Accordingly, in some of these embodiments, wound bed closure may be achieved without changing the dressing.

In some embodiments, the woven meshes described herein are used in wound dressings including negative pressure wound dressings. In other embodiments, the dressing include between 2 and about 10 layers of woven meshes.

In still other embodiments, the woven meshes comprise identical threads. In still other embodiments, the woven meshes comprise different threads.

In some embodiments, the woven meshes are between about 1 mm and about 2 mm thick when dry. In other embodiments, the woven meshes are between about 2 mm and about 4 mm thick when dry.

In some embodiments, the pore size of the woven mesh is between about 1 mm and about 10 mm in width. In other embodiments, the pore size of the woven mesh is between about 0.3 mm and about 0.6 mm in width. In still other embodiments, the pores of the woven mesh are aligned. In still other embodiments, the pores of the woven mesh are staggered. In still other embodiments, the woven meshes are collimated to create pores of desired size.

In some embodiments, the woven mesh is mechanically stable at a minimum vacuum level of about 75 mm Hg. In other embodiments, the woven mesh is mechanically stable at a vacuum up to about 150 mm Hg.

In some embodiments, the woven mesh includes collagen. In other embodiments, the dressing is attached to a polyurethane foam. In still other embodiments, the polyurethane foam is open celled. In still other embodiments, the dressing is attached to a thin film. In still other embodiments, the thin film is silicone or polyurethane. In still other embodiments, the dressing is attached to the thin film with a water soluble adhesive.

In some embodiments, the thread used in the dressing includes a therapeutic agent or a diagnostic agent.

In some embodiments, a negative pressure wound dressing (Johnson et al., U.S. Pat. No. 7,070,584, Kemp et al., U.S. Pat. No. 5,256,418, Chatelier et al., U.S. Pat. No. 5,449,383, Bennet et al., U.S. Pat. No. 5,578,662, Yasukawa et al., U.S. Pat. Nos. 5,629,186 and 5,780,281 and Ser. No. 08/951,832) is provided for use in vacuum induced healing of wounds, particularly open surface wounds (Zamierski U.S. Pat. Nos. 4,969,880, 5,100,396, 5,261,893, 5,527,293 and 6,071,267 and Argenta et al., U.S. Pat. Nos. 5,636,643 and 5,645,081). The dressing includes a pad which conforms to the wound location, an air-tight seal which is removably adhered to the pad, a negative pressure source in fluid communication with the pad and the threads, braids, cords, woven meshes or three-dimensional structures described herein attached to the wound contacting surface of the pad. The pad, seal and vacuum source are implemented as described in the prior art.

In other embodiments, the threads, braids, cords, woven meshes or three-dimensional structures described herein are mechanically stable at a minimum vacuum level of about 75 mm Hg. In still other embodiments, the threads, braids, cords, woven meshes or three-dimensional structures described herein are mechanically stable at a vacuum up to about 150 mm Hg. In still other embodiments, the dressing includes at least one layer of woven mesh. In still other embodiments, the dressing include between 2 and about 10 layers of woven mesh.

In some embodiments a tube connects the pad to the negative pressure source. In still other embodiments, a removable canister is inserted between the pad and the negative pressure source and is in fluid communication with both the pad and the negative pressure source.

In some embodiments, the threads, braids, cords, woven meshes or three-dimensional structures described herein are not hydrated. Accordingly, in these embodiments, the dressing could absorb wound exudates when placed in contact with the wound. In other embodiments, the threads, braids, cords, woven meshes or three-dimensional structures described herein are hydrated. Accordingly, in these embodiments, the dressing could keep the wound moist when placed in contact with the wound.

In some embodiments, an input port attached to a fluid is connected with the pad. Accordingly, in these embodiments, fluid could be dispensed in the wound. In some embodiments, the fluid is saline. In other embodiments, the fluid contains diagnostic or therapeutic agents.

In some embodiments, the threads, braids, cords, woven meshes or three-dimensional structures described herein are used as adhesion barriers. In some embodiments, the woven meshes described herein are used in adhesion barriers.

### Hair Loss Treatment

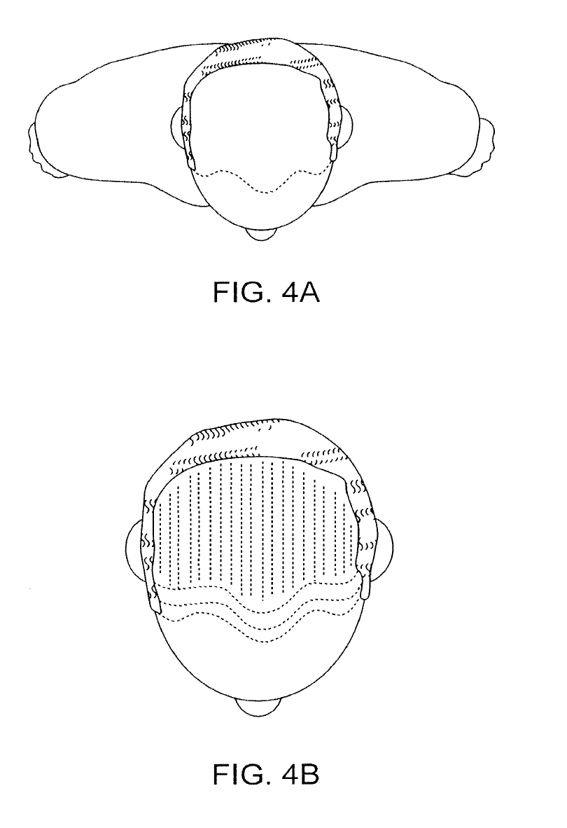

In some embodiments, a method of treating hair loss in a subject is provided. A subject such as, for example, a male with typical male-pattern baldness is illustrated in FIG. 4A and the area where hair growth (with imaginary hairlines) is desired is shown in FIG. 4B. The thread may be attached to a needle as illustrated, for example, in FIGS. 1, 2A, 2B and 2C. The distal end of the needle may be inserted into one of the hair lines as illustrated, for example, in FIG. 4C. The needle then may traverse the area beneath the hairline of the subject and then may exit the skin of the subject as illustrated, for example, in FIG. 4D. The needle may then be pulled distally until it is removed from the subject such that the thread is pulled into the location previously occupied by the needle as illustrated, for example, in FIG. 4E. Finally, excess thread is cut from the needle at the skin surface of the subject which leaves the thread implanted as illustrated, for example, in FIG. 4D.

### Additional Medical and Surgical Treatments

In some embodiments, the threads, braids, cords, woven meshes or three-dimensional structures described herein are used as soft tissue augmentation products in various aesthetic applications. In other embodiments, the threads, braids, cords, woven meshes or three-dimensional structures described herein are used as sutures in various surgical applications. In still other embodiments, the threads, braids, cords, woven meshes or three-dimensional structures described herein are used in ophthalmologic surgery, drug delivery and intra-articular injection.

In some embodiments, a method for treating tumors in a subject in need thereof is provided. The thread may be attached to a needle as illustrated, for example, in FIGS. 1, 2A and 2B. The distal end of the needle may be inserted into the tumor of the subject. The needle then may traverse the tumor and then may exit the tumor. The needle may then be pulled distally until it is removed from the tumor of the subject such that the thread is pulled into the location previously occupied by the needle. Finally, excess thread is cut from the needle which leaves the thread implanted in the tumor of the subject. In some of the above embodiments, the thread includes an anti-cancer agent. In some embodiments, the thread is cross-linked and includes Bcl-2 inhibitors.

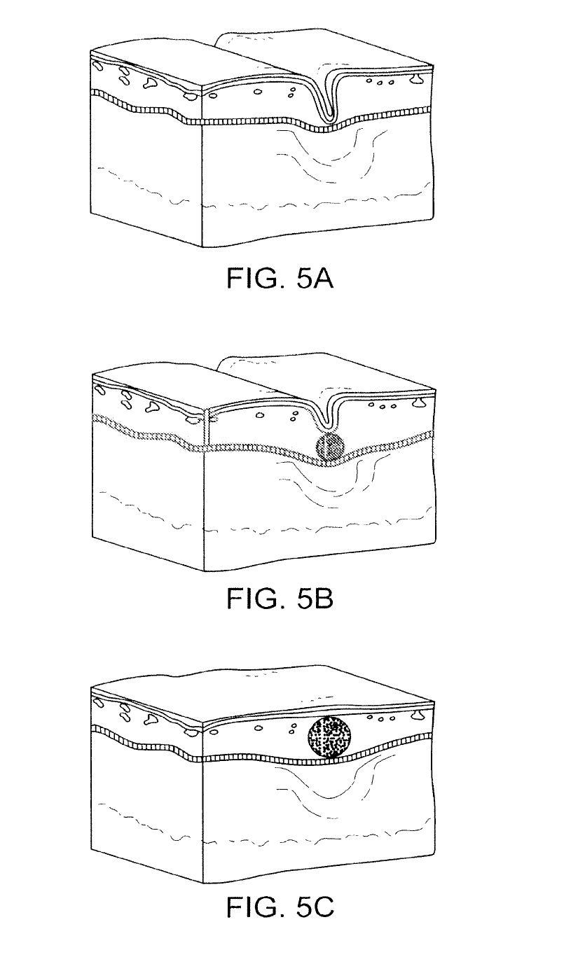

In an exemplary embodiment, methods may be used to treat pancreatic tumors. FIG. 6A illustrates a human pancreas with a tumor while FIG. 6B illustrates a needle with a thread attached thereto. The pancreas may be accessed by surgery or minimally invasively methods such as by laparoscopy. The distal end of the needle may be inserted into the pancreatic tumor. The needle then may traverse the pancreatic tumor as illustrated in FIG. 6C and then may exit the tumor. The needle may then be pulled distally until it is removed from the pancreatic tumor such that the thread is pulled into the location previously occupied by the needle. Finally, excess thread is cut from the needle which leaves the thread implanted in the pancreatic tumor. The process may be repeated any number of times to provide, as illustrated in FIG. 6D, a pancreatic tumor which has been implanted with a number of threads. In some embodiments, the thread includes an anti-cancer agent.

In some embodiments, a method for treating a varicose vein in subject in need thereof is provided. The thread may be attached to a needle as illustrated, for example, in FIGS. 1, 2A and 2B. The distal end of the needle may be inserted into the varicose vein of the subject. The needle then may traverse the varicose vein and then may exit the vein. The needle may then be pulled distally until it is removed from the varicose vein of the subject such that the thread is pulled into the location previously occupied by the needle. Finally, excess thread is cut from the needle which leaves the thread implanted in the varicose vein of the subject. In some embodiments, the needle is a flexible. In other embodiments, the thread coils when hydrated, more readily occluding the vessel.

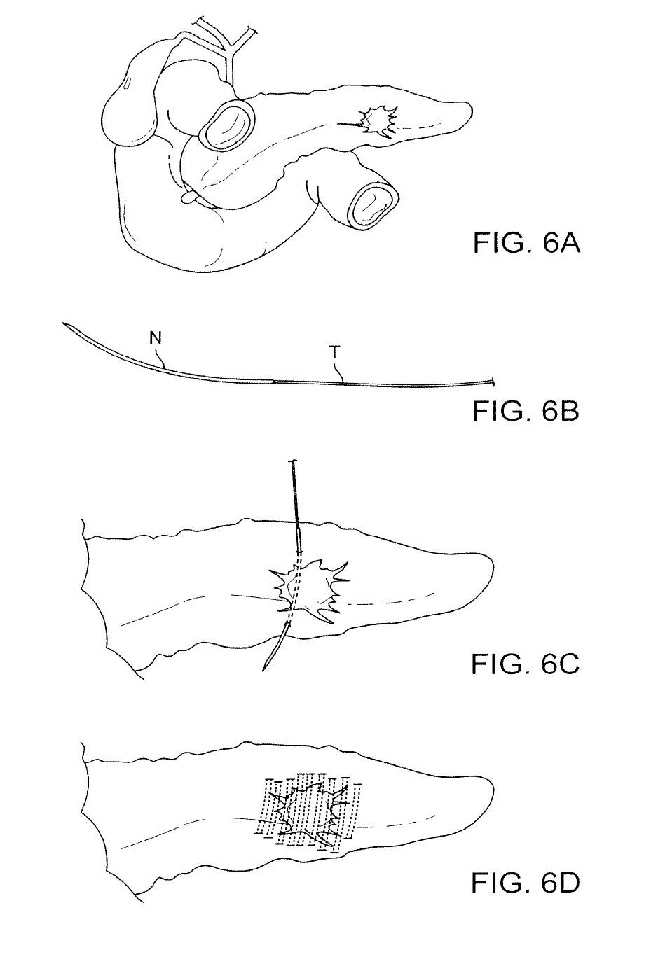

In some embodiments, a method for nipple reconstruction is provided where a three-dimensional, cylindrical implant comprised of cross-linked threads is implanted underneath the skin. The implant may include therapeutic agents, for example chrondrocyte adhesion compounds. FIG. 7A illustrates an implant of multiple layers of concentric coils of threads shaped to represent a nipple while FIG. 7B shows a cross-section of the implant of FIG. 7A. FIG. 7C illustrates how the implant of FIG. 7A could be used for nipple reconstruction.

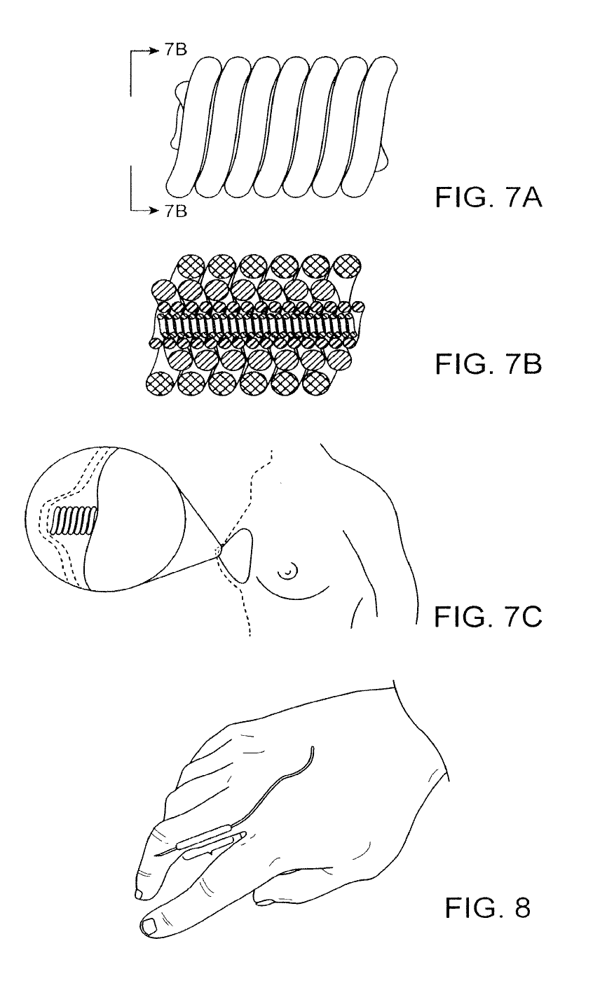

In some embodiments, methods for nerve or vessel regrowth are provided. As illustrated in FIG. 8, a needle can be used to place a thread in a specific line which could promote nerve or vessel regeneration.

### 6. KITS

Also proved herein is a kit of parts comprising a thread. In some embodiments, the kit comprises a thread and a means for delivering or implanting the thread to a patient. In one embodiment, the means for delivery to a patient is a syringe or a needle. In another embodiment, the means for delivery to a patient is an air gun. The size (or diameter) of the needle may depend on the use of the thread, and therefore also be based on the cross-sectional area of the thread used. The outer diameter of the needle or syringe may be greater than or equal to the cross-sectional area of the thread used to lessen the tensile requirement of the thread as it is being applied to the skin. It is further contemplated that the outer diameter of the thread may be larger than the outer diameter of the needle. Skin is quite pliable so by having a smaller diameter needle can allow the puncture size to be small even with the use of a larger diameter thread. Further, the thickness of the thread would be different in the case where the thread is a suture in comparison to the treatment of fine lines and wrinkles where it may be that a thinner thread is used. More than one thread may also be attached to a single needle.

Further, the size of the delivery device, a needle, will be dependent on its intended use and the size of the thread. It is contemplated that for use in facial contouring and or wrinkle filling a 0.006 to about 0.008″ diameter thread or a 0.003 to about 0.004″ diameter thread will be sufficient. In one embodiment, the needle is stainless steel. In other embodiments, the size of the thread is from about 0.01″ to 0.02″ in diameter.

The thread attachment to the needle can be either a mechanical attachment and/or with the use of an adhesive, such as cyanoacrylate. In one embodiment, the thread woven or looped through holes in the proximal end of the needle, or alternatively, the thread wrapped around the proximal end of the needle, or alternatively, the thread threaded thru an eyelet of the needle and either tied or bonded with an adhesive to form a loop, or alternatively, the thread secured (either mechanically or bonded with an adhesive) within a hole in the proximal end of the needle. In another embodiment, the thread can be made to form a physical attachment to the needle during the drying process as the thread forms from the gel. For example, if a needle is used which has pores in the proximal end, the pores can fill with the gel during the extrusion process and the thread would be thus be secured upon drying. The needle can be rigid or flexible to enable the user to track the needle under the wrinkle within the skin. Further, the needle may be equipped with a ramp to guide the needle at a desired depth within the skin, and after needle insertion, the guide may be unclasped as the needle is brought through the skin surface. In some embodiments, the thread is attached to a needle.

It is further contemplated that the kit comprises a needle and the thread attached thereto, is packaged sterile, and intended for single use. Alternatively, a kit can comprise several needles, each with an attached thread. In an additional embodiment, a kit includes threads of different sizes to enable treatment options for the physician while minimizing the number of required needle sticks. In yet another embodiment, the kit includes threads and needles of different length and curved shapes to simplify implantation in areas that are difficult to access or treat with a straight needle, for example near the nose, around the eyes and the middle portion of the upper lip.

### EXAMPLES

The present disclosure is further defined by reference to the following examples. It will be apparent to those skilled in the art that many modifications, both to threads and methods, may be practiced without departing from the scope of the current disclosure. The biocompatible polymers and other reagents (i.e., cross-linking agents) are available from commercial sources.

### Example 1

### Alginate Thread

Sodium alginate (8 grams, molecular weight 10,000-100,000, Acros Organics) was dissolved in 92 grams of double distilled water and stirred for one hour. The gel was incubated at 4 degrees for another 6 hours. A thread was extruded using an extruder over a 4% calcium chloride solution spread on Teflon film. By controlling the flow rates of both the alginate stream and the extruder velocity, uniform threads were prepared. The semi-dry threads were then stretched and dried. The results of the failure stress test for a dry thread and a wet thread are shown below.

| | Dry test | Wet |
|---|---|---|
| Run | (in pounds) | (in pounds) |
| 1 | 0.7994 | 0.19 |
| 2 | 0.9072 | 0.1934 |
| 3 | 0.6856 | 0.1446 |
| 4 | 0.928 | 0.2606 |
| Average | 0.83005 | 0.19715 |

### Example 2

### Chitosan Thread

Chitosan (8 grams) was dissolved in 20 mL acetic acid in water (2% vol/vol), extruded on a Teflon sheet, and dried to provide a dry thread. The dry threads were then wetted with water and subjected to freeze-drying using a freeze dryer. The porous lyophilized threads were soaked in a solution containing sodium trimetaphosphate (19 g), ca 0.75 g NaOH (pellets) and 150 ml MilliQ water. The threads were allowed to soak in the solution for 4-6 hours and washed with double distilled water until the pH was about 7. The threads are then air dried again. The results of the failure stress test for a dry thread are shown below.

| | Dry test | |
|---|---|---|
| Run | (in pounds) | Extension |
| 1 | 0.6354 | 0.774 |
| 2 | 0.799 | 0.753 |
| Average | 0.7172 | 0.7635 |

### Example 3

### Synthesis of a Thread

A soft tissue augmentation thread of a diameter of up to 1 mm can be made by the following procedure. It is contemplated that a thread as prepared below can be stored under ambient conditions for greater than 9 months without a loss of its structural integrity.
1. The desired amount of a biocompatible polymer is weighed out into a suitable container and an aqueous solution, such as deionized water, is added to result in the desired % biocompatible polymer gel by weight.
2. The biocompatible polymer is allowed to dissolve in the aqueous solution at a temperature of about 4-10° C. for 8 to 24 hours until the biocompatible polymer has completely swelled thus forming a gel. With higher molecular weight biocompatible polymers (e.g. >2 MDa) and/or higher % gels (e.g. >10%), a longer swelling time may be required, or alternatively, the composition can me mechanically stirred. The viscosity of the gel composition is typically from about 150 Pascal-seconds (Pa·s) to about 2,000 Pascal-seconds (Pa·s). Optionally, the gel can be degassed by applying a vacuum or by freeze-pump-thaw cycles.
3. The gel composition is then transferred to a pressurized extruder (e.g., EFD Model XL1500 pneumatic dispense machine). The nozzle of the extruder can have a tip ranging from a 15 gauge to about 25 gauge. The syringe pressure may be between about 10 psi and about 2,000 psi, depending on the viscosity of the gel composition. For very viscous gels, a pressure multiplier can be used.
4. The wet thread is then formed by extruding the gel composition onto a substrate by an extruder which is linearly translating at a speed commensurate with the speed of gel ejection from the syringe to achieve the desired wet thread thickness.
5. The wet thread is then dried under ambient conditions for about 12 hours to a percent moisture of less than about 30%, or less than about 15%, or less than about 10%, thus providing a dry thread.
6. Optionally, a desired amount of cross-linking agent (e.g., 2% by weight) can be added to the aqueous solution of step 2 or to the wet thread of step 4.
7. Optionally, prior to or during step 5, the wet thread can be stretched to a desired length and reduced diameter prior to dying. The stretching can be by either hanging the thread by one end and applying weight to the opposing end, or by horizontally stretching the wet thread on a surface (either the same or different from the extrusion surface) and adhering or tying the thread ends to the surface.

### Example 4

### Washing (Re-Hydrating) and Re-Drying the Thread

The dry threads can then be washed with an aqueous solvent to remove any contaminants such as, for example, unreacted cross-linking agent. The washing can be performed by various methods, such as submersion in an aqueous solvent or by using a concurrent flow system by placing the thread in a trough at an incline and allowing an aqueous solvent to flow over the thread. In addition, the thread, once it is rehydrated, can be stretched prior to re-dying. The stretching can be performed by the means described above in Example 3. The rehydrated and washed thread is then re-dried to provide the dry thread. The re-drying is typically performed under ambient conditions (i.e. ambient temperature and/or pressure) for from about 8 hours to about 24 hours or until the dry thread has a percent moisture of less than about 30%. The thread can be washed several times (e.g. 10 or more times) without losing its structural integrity. Over the course of multiple washing cycles the overall length of the thread can be increased by between about 25% and about 100%.

### Example 4

### Determination of Ultimate Tensile Strength of Dermal Filler Threads

Various threads prepared as described above can be tested for tensile strength using a force gauge (e.g. Digital Force Gauge by Precision Instruments). A zero measurement is the result of an inability to form a thread of testing quality.

### Example 5

### Treatment of Wrinkles of a Cadaver with Dermal Filler Threads

Hypodermic needles (22 Ga) are affixed with single or double strands of soft tissue augmentation threads with super glue (e.g., LocTite 4014). The needles are able to traverse wrinkles in a cadaveric head such as the naso-labial fold, peri-orals, peri-orbitals, frontalis (forehead), and glabellar. The needle pulls the thread through the skin such that the thread is located where the needle was previously inserted. More than one thread can be used to treat the wrinkles in order to achieve the desired fill effect (e.g., two or more threads). Since cadaveric tissue does not have the same hydration characteristics as living tissue, the threads are hydrated by applying a 0.9% saline solution to the treated area. The treated wrinkle is visibly lessened upon thread hydration.

### Example 6

### Organization of the Threads Via Atomic Force Microscopy (AFM)

The organization in the threads can be determined by atomic force microscopy (AFM) when compared to the gel composition before the thread is formed. The AFM images can be collected using a NanoScope III Dimension 5000 (Digital Instruments, Santa Barbara, Calif., USA). The instrument is calibrated against a NIST traceable standard. NanoProbe® silicon tips are used. Image processing procedures involving auto-flattening, plane fitting or convolution can be employed. One appropriately sized area can be imaged at a random location for both the gel and the thread samples. The topography differences of these images can be presented in degree of shading where the dark areas are low and the light areas are high. AFM images and the Phase image are acquired simultaneously. The roughness analyses can be performed and are expressed in: (1) Root-Mean-Square Roughness, RMS; (2) Mean Roughness, Rₐ; and (3) Maximum Height (Peak-to-Valley), Rₘₐₓ. The phase image monitors differences in the interaction of the tip with the sample which can be induced by composition and/or hardness differences.

### Example 7

### In Vitro or In Vivo Testing Regarding Increase in Fibrogenesis

The in vivo stimulation of collagen production caused by the threads can be accomplished using methods known in the art. For example, according to the methods of Wang et al. (Arch Dermatol. (2007) 143(2):155-163), the thread can be applied to a patient followed by a biopsy of the treatment area at one or more time intervals following treatment. The de novo synthesis of collagen can then be assessed using immunohistochemical analysis, quantitative polymerase chain reaction, and electron microscopy.

### Example 8

### Water Content of Dry Threads by Karl Fisher Titration

Threads made by the methods above can be tested for the percent moisture via Karl Fisher titration.

### Example 9

### Organization of the Threads Via Transmission Electron Microscopy (TEM)

Samples of biocompatible polymer gel and thread as prepared by the methods above can be removed from refrigerator then capped with protective carbon, iridium metal, and local platinum. TEM-ready samples can then be prepared by focused ion beam (FIB) milling. The fiber samples can be cross sectioned in the longitudinal direction using the in situ FIB lift out method with a FEI 830 Dual Beam FIB fitted with an Omniprobe Autoprobe 2000. The gel sample can be a random cut. TEM imaging can be performed at room temperature in bright-field TEM mode using a FEI Tecnai TF-20 operated at 200 kV.

### Example 10

### Lip Augmentation

A patient can be implanted with soft tissue augmentation threads for lip enhancement, either contouring and/or plumping. The patient receives only topical anesthetic on the face, but it is not applied specifically to the lips. The following procedure is followed:
Peal open the pouch and remove the sterile tray holding the soft tissue augmentation threads.
Using sterile gloves or a sterile implement such as forceps, remove the desired soft tissue augmentation thread from the tray.
Insert the sharp end of the needle into one margin of the intended treatment area.
Translate the needle within the skin under or near the intended treatment area. If the needle is not in a desired location at any point, gently retract the needle and reinsert to correct the location.
Exit the skin at the opposing margin of the intended treatment area using the sharp end of the needle. If the needle is not in the desired location, gently retract the needle and reinsert to correct the location.
Upon confirming the desirable location of the needle, swiftly pull the needle distally, pulling the thread into place within the skin.
Using sterile surgical scissors or scalpel, cut the excess thread protruding from the skin on both margins of the treatment area. This effectively separates the needle, which should be discarded appropriately.

Areas of enhancement include the vermillion border (or white roll) for lip effacement and contouring, the wet-dry mucosal junction for increasing fullness. Other techniques include more diffuse infiltration of the orbicularis oris muscle. The attending clinician is able to select the location of the thread placement, the number of threads and the size of the threads depending on desired effect. It is contemplated that each area is treated with 1 to 2 threads wherein each thread has a diameter of anywhere from 200 microns to about 500 microns when the thread is dry. After hydration, it is contemplated that the thread is from 0.5 millimeters to about 5 millimeters.

It will be appreciated that those skilled in the art will be able to devise various arrangements which, although not explicitly described or shown herein, embody the principles and are included within its spirit and scope. Furthermore, all conditional language recited herein is principally intended to aid the reader in understanding the principles and the concepts contributed by the inventors to furthering the art, and are to be construed as being without limitation to such specifically recited conditions. Moreover, all statements herein reciting principles, aspects, and embodiments are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

What is claimed is:

1. A method of improving the appearance of the face, the method comprising:
implanting a swellable, hyaluronic acid based thread beneath a wrinkle or fold in the face.

2. The method of claim 1 wherein the thread has a tensile strength of about 5 kpsi or greater.

3. The method of claim 1, wherein the thread has a failure of stress of 0.5 pounds or greater.

4. The method of claim 1 wherein the thread has a diameter from 0.006″ to about 0.008″.

5. The method of claim 1 wherein the thread has a diameter from 0.003 to about 0.004″.

6. The method of claim 1 wherein the thread has a diameter from about 0.01″ to 0.02″.

7. The method of claim 1 wherein the thread has a diameter from 0.004″ to about 0 0.008″.

8. The method of claim 1 wherein the step of implanting comprising implanting more than one swellable, hyaluronic acid based thread beneath the nasolabial fold.

9. The method of claim 1 wherein the step of implanting comprises implanting at least two swellable, hyaluronic acid based threads beneath the nasolabial fold.

10. The method of claim 1 wherein the step of implanting comprises implanting three swellable, hyaluronic acid based threads beneath the nasolabial fold.

11. The method of claim 1 wherein the step of implanting comprises implanting four swellable, hyaluronic acid based threads beneath the nasolabial fold.

12. The method of claim 1 wherein the step of implanting comprises implanting the at least two swellable, hyaluronic acid based threads beneath the nasolabial fold in a parallel orientation to one another.

13. The method of claim 1 wherein the thread is cross-linked with a cross-linking agent selected from the group consisting of butanediol diglycidyl ether (BDDE), divinyl sulfone (DVS), and 1-ethyl-3-(3-dimethylaminopropyl) carbodimide hydrochloride (EDC), or a combination thereof.

14. The method of claim 1 wherein the cross-linking agent is butanediol diglycidyl ether (BDDE).

15. A method of improving the appearance of the face, the method comprising:
filling in a wrinkle on the face by implanting a swellable, hyaluronic acid based thread beneath the wrinkle, the thread being inserted adjacent to or under the wrinkle; and
allowing the thread to expand by becoming hydrated upon exposure to body fluids the expansion being sufficient to lessen the appearance of the wrinkle.

16. The method of claim 15 wherein the wrinkle is a wrinkle selected from a nasolabial fold and a forehead wrinkle.

17. The method of claim 15 wherein the thread has a diameter of about from about 0.004″ to about 0.0.008″ in diameter.

18. The method of claim 15 wherein the thread has a diameter from 0.003 to about 0.004″.

19. The method of claim 15 wherein the thread has a diameter from about 0.01″ to 0.02″.
